(19) United States
(12) Plant Patent Application Publication
(10) Pub. No.: US 2003/0097698 P1
(43) Pub. Date: May 22, 2003

(54) PETUNIA PLANT NAMED 'KAKEGAWA S59'

(76) Inventor: Masao Bessho, Kakegawa City (JP)

Correspondence Address: JONDLE & ASSOCIATES P.C., 9085 EAST MINERAL CIRCLE, SUITE 200, CENTENNIAL, CO, 80112, US

(21) Appl. No.: 09/989,537

(22) Filed: Nov. 21, 2001

(51) Int. Cl. A01H 5/00

(52) U.S. Cl. PLT/356

(57) ABSTRACT

A Petunia cultivar particularly distinguished by its pink flower color and creeping, mounding habit.

### BACKGROUND OF THE NEW PLANT

The present invention comprises a new and distinct cultivar of Petunia, botanically known as Petunia hybrida, and hereinafter referred to by the cultivar name ‘Kakegawa S59’. ‘Kakegawa S59’ originated from a hybridization made in 1997 in Kakegawa, Japan. The male parent originated from a cross made in 1992 between a dwarf multiflora pink breeding line and a small, magenta flowered breeding line with creeping habit. From a total of twenty F₁ plants produced, three were selected in 1993 for intercrossing. From a total of 80 F₂ plants, one plant with small, light pink flowers and a creeping habit was selected in fall 1993 as line 4-185F-1a. In spring 1994, 4-185F-1a was crossed to a dwarf multiflora pink breeding line known as 9S-829-2a-1a-1a-1. Three plants were selected from the F₁ seed and intercrossed to produce F₂ seed. In 1995 a large, pink flowered plant with creeping habit was selected and over the next two years single plant selection was used to self the male parent to the F₄ generation. In 1997, ‘Kakegawa S59’s' male parent was crossed again to the female parent, dwarf multiflora pink breeding line 9S-829-2a-1a-1a-1.

Two hundred F₁ plants were transplanted to the field in Salinas, Calif. during the summer of 1998. Four lines were selected for further evaluation and vegetatively propagated. The four lines were propagated again in 1999 and evaluated for fixed characteristics and ease of propagation. Final selection of one line was made in Salinas, Calif. during the summer of 1999. The line was established as ‘Kakegawa S59’, and determined to have its characteristics firmly fixed.

‘Kakegawa S59’ has been found to retain its distinctive characteristics after two years and four cycles of vegetative propagation and this novelty is firmly fixed. The variety has demonstrated stability during this time and has no inherent variation or off-types.

### DESCRIPTION OF PHOTOGRAPH

This new Petunia plant is illustrated by the accompanying photograph which shows blooms, and foliage of the plant in full color, the colors shown being as true as can be reasonably obtained by conventional photographic procedures.

FIG. 1 shows the mature inflorescence;

FIG. 2 shows the entire plant approximately eight weeks after transplanting a rooted cutting.

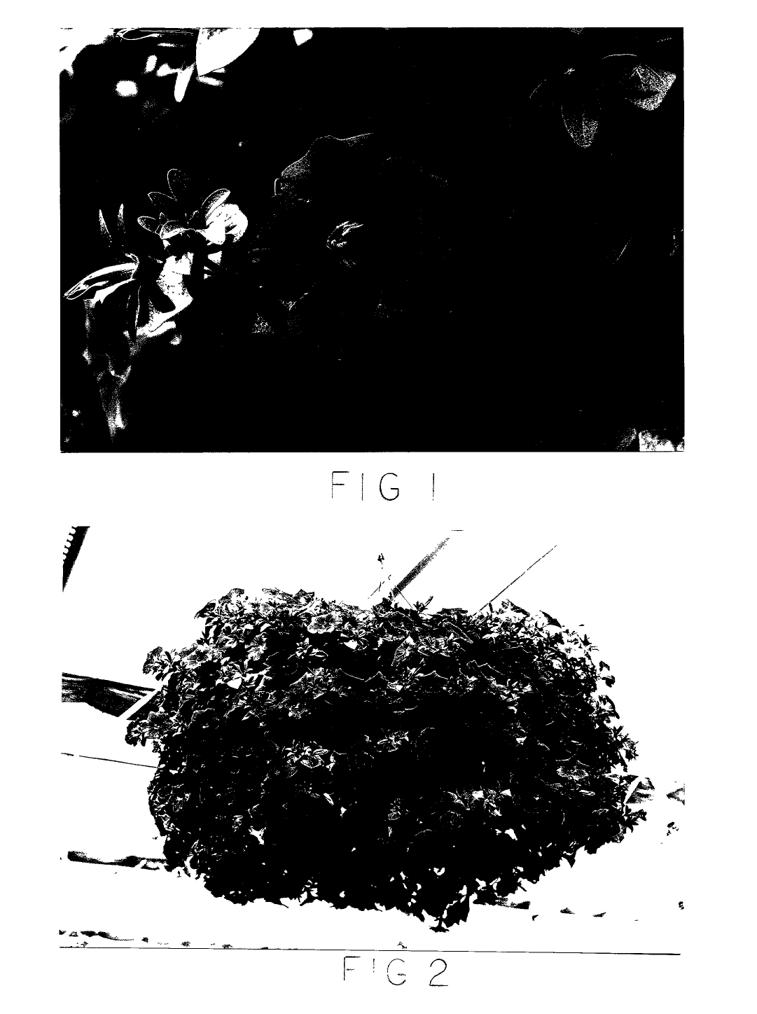

### DESCRIPTION OF THE NEW CULTIVAR

The following detailed descriptions set forth the distinctive characteristics of ‘Kakegawa S59’. The data which defines these characteristics were collected from asexual reproductions carried out in Salinas, Calif. Three plants from fully rooted 15 cm diameter pots were transplanted to one 50 cm diameter hanging baskets and grown in the same conditions. Data was collected on plants in 50 cm diameter pots eight weeks after rooted cuttings were transplanted. Color references are primarily to the RHS Colour Chart of The Royal Horticultural Society of London (RHS).

### DESCRIPTION OF THE NEW PLANT

Classification:

Botanical.—Petunia hybrida.

Commercial.—Petunia.

Parentage:

Female parent.—breeding line 9S-829-2a-1a-1a-1 (not patented).

Male parent.—an F₄ selection from a cross between breeding lines 92-829-2a-1a-1a-1 (not patented) and 4-185F-1a (not patented).

Environmental Conditions for Plant Growth: Plants were propagated from vegetative cuttings, and grown individually in 15 cm diameter plastic pots in a glass greenhouse located in Salinas, Calif. Pots contained a peat moss-based growing medium. Soluble fertilizer containing 18% nitrogen, 8% phosphorus and 18% potassium was applied in four, daily irrigations. The fifth irrigation was made with non-fertilized water. Pots were top-dressed with a slow release fertilizer containing 18% nitrogen, 8% phosphorus and 18% potassium. The typical average air temperature was 24C.

Growth:

Habit.—branching, creeping.

Form.—descending.

Plant size.—91 cm total diameter and 76 cm total height when three plants are grown in a 50 cm diameter hanging basket for 7-8 weeks.

Flowering habit.—indeterminate.

Time to bloom from propagation.—4-6 weeks after rooting when grown in 10-15 cm diameter plastic pots.

Life cycle.—annual.

Stems:

Color.—yellow-green (RHS 146A).

Description.—round.

Diameter.—0.4 to 0.5 mm.

Internode length.—3.0-4.0 cm.

Leaves:

Arrangement.—opposite.

Apex.—mucronate.

Base.—oblique.

Color.—upper surface is green (RHS 139B) and lower surface is green (RHS 138C).

Margin.—entire.

Size.—length is 4.8 cm and width is 4.5-5.0 cm.

Shape.—ovate.

Texture.—coarse.

Venation.—pinnate.

Pubescence.—present, clear.

Flowers:

Calyx.—5 sepals; 2.3 cm×0.5 cm (length×width).

Corolla.—5 petals, fused.

Flower diameter.—6.0-7.0 cm.

Fragrant.—yes.

Inflorescence type.—solitary.

Pistil.—compound.

Ovary.—superior, parietal placentation.

Stamens.—5 total with two long and three short.

Peduncle.—2.5-3.0 cm×0.2 mm (length×width); pubescent.

Petal color.—Limbs: upper—red-purple (RHS 74B); lower—red-purple (RHS 73A). Tube—Inner—red-purple (RHS 73C); Outside is red-purple (RHS 69C) with green (RHS 141C) veins.

Petal pubescence.—absent.

Tube throat diameter.—1.0 cm.

Pollen color.—White (RHS 155A).

Produces seed.—Yes.

Disease and Insect Resistance No susceptibility to diseases or insects noted to date.

Comparison with Known Cultivars ‘Kakegawa S59’ is most similar to the variety ‘Cascadia Pink’ but differs in the following areas: ‘Kakegawa S59’ has a true pink petal limb color where ‘Cascadia Pink’ is a more reddish color. The inside color for the corolla tube is white on ‘Cascadia Pink’ where it is a light pink on ‘Kakegawa S59’.

I claim:

1. A new and distinct cultivar of Petunia as shown and described herein.
